(19) United States
(12) Patent Application Publication
(10) Pub. No.: US 2020/0064351 A1
(43) Pub. Date: Feb. 27, 2020

(54) POINT-OF-CARE DEVICE FOR THE QUANTITATIVE DETERMINATION OF BRANCHED-CHAIN AMINO ACIDS IN BIOLOGICAL SAMPLES

(71) Applicant: ANALYTICAL DIAGNOSTIC SOLUTIONS, INC., Mount Laurel, NJ (US)

(72) Inventors: Robert Harper, Mount Laurel, NJ (US); Jordan T. Seville, Delran, NJ (US)

(21) Appl. No.: 16/610,619

(22) PCT Filed: May 4, 2018
(86) PCT No.: PCT/US18/31032

§ 371 Date: Nov. 4, 2019

Related U.S. Application Data
(60) Provisional application No. 62/502,031, filed on May 5, 2017.

(51) Int. Cl. G01N 33/68; B01L 3/00; G01N 33/558; G01N 31/22

(52) U.S. Cl. CPC G01N 33/6812; B01L 3/50; B01L 2300/0825; G01N 31/22; G01N 33/558

(57) ABSTRACT

Devices, kits, and assays are provided for the testing and monitoring of branched-chain amino acids in biological fluids.

This patent application claims the benefit of priority from U.S. Provisional Application Ser. No. 62/502,031 filed May 5, 2017, the contents of which is herein incorporated by reference in its entirety.

### FIELD OF THE INVENTION

The present invention relates to devices, kits, and assays for the testing and monitoring of branched-chain amino acids (BCAAs) in biological samples.

### BACKGROUND OF THE INVENTION

Maple syrup urine disease (MSUD), also known as branched-chain ketoaciduria is a rare genetic disorder characterized by mutations in one or more of the four interconnected proteins in the branched-chain alpha-keto acid dehydrogenase complex which is essential for breaking down the amino acids L-leucine, L-isoleucine, L-alloisoleucine, and L-valine. When these amino acids are not effectively metabolized, they can accumulate in the cells and fluids of the body. Such accumulation can cause a variety of symptoms including lethargy, irritability, poor feeding, abnormal movements and a characteristic odor similar to that of maple syrup in the earwax (cerumen), sweat, and urine of affected individuals. In addition, if untreated various neurological complications including seizures, coma, and brain damage may occur. Failure to promptly detect and treat MSUD can lead to life-threatening complications.

The onset, symptoms, and severity of MSUD varies greatly from case to case. Four distinct clinical forms of the disorder have been identified: classical, intermediate, intermittent, and thiamine-responsive. Some researchers include dihydrolipoyl dehydrogenase (E3)-deficient MSUD as a fifth subtype. The severity of MSUD relates to the amount of residual enzyme activity in the body.

Classical maple syrup urine disease is the most common and most severe form of MSUD. There is little to no enzyme activity present. Most infants with classic MSUD will show symptoms within the first few days of life. Breastfeeding may delay symptoms into the second week of life. Such symptoms may include lethargy, irritability, failure to thrive, poor sucking response, and little interest in feeding. The distinctive odor of maple syrup may be detected in earwax (cerumen), sweat, and urine. Additional symptoms may develop including weight loss, irregular sleep patterns (intermittent apnea), and episodes of abnormal muscle rigidity (hypertonia) alternating with periods of extreme floppiness (hypotonia). Eventually, affected infants experience seizures, coma, and if left untreated, neurological damage including mental retardation and life-threatening complications such as central respiratory failure.

With proper treatment utilizing BCAA-free medical foods, a reduced BCAA/leucine diet, thiamine supplementation, and/or isoleucine and valine supplementation, affected infants can experience normal growth and development. However, there is a continued risk of symptomatic episodes known as metabolic crisis, or metabolic decompensation. These episodes are characterized by a sudden and often rapid increase in branched-chain amino acids in the blood and cells of the body. Episodes may be triggered by infection, psychological stress, injury, or failure to eat (fasting). In infants, associated symptoms may include altered consciousness, a group of movement disorders characterized by involuntary muscle contractions that force the body into abnormal or painful movements and positions (dystonia), and an inability to coordinate voluntary movements (ataxia). Severe episodes can result in coma, swelling of the brain due to fluid accumulation (edema), brain damage due to lack of blood flow (ischemia) to the brain, mental retardation, and other life-threatening complications such as central respiratory failure.

Many cases of MSUD are detected through newborn screening programs. Heel-stick samples are taken and whole blood is spotted on filter paper and sent out to laboratories for analysis by tandem mass spectrometry. Tandem mass spectrometry, an advanced newborn screening test that allows physicians to test for more than 30 different disorders through one blood sample, has aided in the diagnosis of MSUD. In areas where testing for MSUD via tandem mass spectrometry is unavailable or where newborn screening fails to detect MSUD, diagnosis may be based upon characteristic findings (e.g., lethargy, failure to thrive, odor of maple syrup in earwax, sweat, or urine). Alternative tests to diagnose MSUD may include urine analysis to detect high levels of ketonic acids (ketoaciduria) and blood analysis to detect abnormally high levels of amino acids. A diagnosis may be confirmed through the analysis of the patient's white blood cells (lymphocytes) or cells taken from an affected individual's skin.

### SUMMARY OF INVENTION

An aspect of the present invention relates to a test strip for the quantitative determination of one or more branched-chain amino acids (BCAAs), including but not limited to L-Valine, L-Leucine, L-Isoleucine, and L-Alloisoleucine in a biological sample.

Another aspect of the present invention relates to diagnostic kits and assays for the colorimetric and quantitative determination of one or more BCAAs in biological samples. The kit and assay comprise a combination of components which elicit a colored end product which is specific for BCAA concentrations in the biological sample.

In one non-limiting embodiment, the kit and assay use a test strip for assaying BCAAs.

In one non-limiting embodiment, the kit and assay require less than 20 μL of a fingerstick sample of blood, urine, or saliva from a collection vessel for analysis.

Another aspect of the present invention relates to methods for identifying individuals with Maple Syrup Urine Disease and other disorders relating to deficiency or excess in BCAAs, as well as monitoring BCAA levels in such individuals.

Another aspect of the present invention relates to monitoring BCAA levels in individuals before, during, and following performance training.

### BRIEF DESCRIPTION OF THE FIGURES

FIG. 1 is a 3^{rd} order polynomial of MS/MS concentration (mg/dL L-Leucine) versus the Percent Reflectance obtained.

FIG. 2 is a first order linear regression of MS/MS concentration (mg/dL L-Leucine) versus interpolated mg/dL L-Leucine from the measured Percent Reflectance.

### DETAILED DESCRIPTION OF THE INVENTION

The present invention provides test strips, kits, and assays for the quantitative determination of branched-chain amino acids (BCAAs), including but not limited to L-Valine, L-Leucine, L-Isoleucine, and L-Alloisoleucine in a biological fluid, including but not limited to whole blood, saliva, and urine, as well as methods for identifying and monitoring individuals with disorders relating to a deficiency or excess in BCAAs and monitoring BCAA levels in such individuals.

The test strips, kits, and assays can be based on visual end-color, percent reflectance, RGB values, piezoelectric methods, electrochemiluminescence methods, electrochemical response, or kinetic determination.

The test strips, kits, and assays provide a quantitative, faster, more rugged, and easier means to perform BCAA analysis as compared to analogous wet chemistry assays, dried blood spot cards, and MS/MS. The test strips, kits, and assays can be used at the point-of-care with, for example, a fingerstick blood sample analogous to glucose testing and can be performed and monitored by either the patient or the clinician.

In one non-limiting embodiment, BCAA concentrations are quantified as either absent, insufficiently present, normal, or in excess within a biological sample.

In one non-limiting embodiment, red and white blood cells of a sample are filtered out through a treated size exclusion membrane, allowing BCCAs residing within the plasma to reach a reagent layer where BCAAs are quantitatively determined colorimetrically using, for example, a reflectance-based reader, photometer, photometric imager, or biosensor.

In one non-limiting embodiment, a test strip is used comprising at least two superimposed membrane layers. In one non-limiting embodiment, the membrane layers can be adhered to a base material made of, for example, polyester through lamination with pressure sensitive adhesives or ultrasonic welding. In another non-limiting embodiment, the membrane layers are superimposed without lamination or adhesive tapes via a clamshell design or stack design. The clamshell and stack design may comprise a pliable plastic housing. The top of the clamshell folds over and clasps or clips onto the base layer, which holds the membranes in place without adhesives or tapes. The plastic holder can also be comprised of a separate lid and base (plastic stack) to incorporate or stack the membranes.

The test strip can be comprised of multiple, stacked membranes or laterally placed membranes which provide a means of transport and filtration for the biological sample which ultimately ends with a reagent layer that gives a detectable, quantitative change or response to the presence of BCAAs within a biological sample applied to the strip. In one non-limiting embodiment, the biological sample is whole blood from, for example, a fingerstick sample. Alternative biological samples which can be used include urine and saliva. In one non-limiting embodiment, a sample of less than 20 μL of biological sample is used.

The test strip of the present invention comprises a sample spreading layer. The sample spreading layer is capable of distributing or metering the cells in a whole blood sample or other biological sample evenly across the surface of a primary membrane. The spreading layer provides a uniform concentration of the cells at the interface of the spreading layer and the primary membrane. Accordingly, precise permeability of the spreading layer is critical to providing uniform distribution of the biological sample across the surface of the primary membrane layer. Examples of materials for the spreading layer include, but are not limited to, hydrophilic mesh materials, isotropic porous materials (same porosity throughout), and anisotropic membranes (having a gradient in porosity and a differential pore size). In one non-limiting embodiment, the spreading layer comprises a hydrophilic mesh with a pore size ranging from 10 to 120 microns. In one non-limiting embodiment, the surface of the spreading layer is in direct contact with the primary membrane for uniform transfer of the biological fluid.

A biological fluid applied to the test strip flows transversely across the spreading layer and then migrates vertically or laterally into the primary membrane. The primary membrane is a blood separation membrane, lateral flow or transport membrane to remove interfering compounds. The blood separation membrane can be composed of, or a blend of, several materials including, but not limited to, glass fiber, polyamide (nylon), polyester, cellulose, nitrocellulose, polysulfone, and polyethersulfone. The primary membrane is further comprised of one or more known hemagglutinating agents including, but not limited to, antibodies, lectins, cationic polymers such as chitosan or diethylaminoethyl-dextran, anionic polymers such as poly(sodium 4-styrenesulfonate), surface treated or magnetic beads, and/or particles/nanoparticles. In one non-limiting embodiment, one or more hemagglutinating agents are immobilized with a mordant such as, but not limited to, hydroxypropyl cellulose, hydroxyethyl cellulose, polyvinyl alcohol, polyvinylpyrrolidone, Poly(1-vinylpyrrolidone-co-vinyl acetate), sodium carboxymethyl cellulose, xanthan gum, and/or hydrogels.

The agglomerated cells are than size excluded out of the biological solution.

The remaining sample is then vertically or horizontally fed into a secondary membrane of the test strip. The secondary membrane, Membrane-2, may be an optional transport membrane, secondary filtering membrane, or reagent membrane. Membrane-2 may be composed of, or a blend of, several materials including, but not limited to, glass fiber, polyamide (nylon), polyester, cellulose, nitrocellulose, polysulfone, and polyethersulfone. Membrane-2 may be further comprised of one or more known hemagglutinating agents including, but not limited to, antibodies, lectins, cationic polymers such as chitosan or diethylaminoethyl-dextran, anionic polymers such as poly(sodium 4-styrenesulfonate), surface treated or magnetic beads, and/or particles/nanoparticles. In one non-limiting embodiment, one or more hemagglutinating agents are immobilized with a mordant such as, but not limited to, hydroxypropyl cellulose, hydroxyethyl cellulose, polyvinyl alcohol, polyvinylpyrrolidone, Poly(1-vinylpyrrolidone-co-vinyl acetate), sodium carboxymethyl cellulose, xanthan gum, and/or hydrogels. In one non-limiting embodiment, Membrane-2 is immobilized with the bioactive components Leucine Dehydrogenase, Nicotinamide adenine dinucleotide (NAD⁺), Diaphorase, and a tetrazolium salt as an indicator.

Membrane-2 may also contain a buffer and stabilizing agents for the preconditioning of the biological sample. The biological fluid is preconditioned with a buffering system, which is immobilized via a mordant onto the membrane. In one non-limiting embodiment, Membrane-2 contains a buffer adjusted in the range of pH 6.0 to pH 10.5.

Examples of mordants which can be used for immobilizing the bioactive components include, but are not limited to, hydroxypropyl cellulose, hydroxyethyl cellulose, polyvinyl alcohol, polyvinylpyrrolidone, Poly(1-vinylpyrrolidone-co-vinyl acetate), sodium carboxymethyl cellulose, xanthan gum, and/or hydrogels.

The biological fluid may then migrate vertically or horizontally into a third membrane. The third membrane, Membrane-3, contains an indicator and bioactive components. In one non-limiting embodiment, Membrane-3 is immobilized with the bioactive components Leucine Dehydrogenase, Nicotinamide adenine dinucleotide (NAD⁺), Diaphorase, and a tetrazolium salt as an indicator. Leucine Dehydrogenase may be derived, for example, from Bacillus cereus or Bacillus stearothermophilus. The Diaphorase may be derived, for example, from Clostridium kluyveri, Porcine Heart, Escherichia coli, Bacillus stearothermophilus, or Bacillus megaterium. Examples of tetrazolium salts include, but are not limited to, WST-1, WST-3, WST-4, WST-5, WST-8, WST-9, WST-10, WST-11, Iodonitrotetrazolium chloride, Thiazolyl Blue tetrazolium bromide, p-Nitroblue Tetrazolium Chloride, Blue Tetrazolium chloride, Neotetrazolium chloride, 2,3-Bis(2-methoxy-4-nitro-5-sulfophenyl)-2H-tetrazolium-5-carboxanilide inner salt, 2,3,5-Triphenyltetrazolium Chloride, 2,3,5-Triphenyltetrazolium Bromide, 5-[3-(Carboxymethoxy)phenyl]-3-(4,5-dimethyl-2-thiazolyl)-2-(4-sulfophenyl)-2H-tetrazolium inner salt, 2,5-Diphenyl-3-(1-naphthyl)tetrazolium chloride, 5-Cyano-2,3-di-(p-tolyl)tetrazolium chloride, Tetranitro blue tetrazolium chloride, and 2-(2′-Benzothiazolyl)-5-styryl-3-(4′-phthalhydrazidyl)tetrazolium chloride.

Leucine Dehydrogenase catalyzes the oxidation of BCAAs (L-leucine, L-valine, L-isoleucine, and L-alloisoleucine) while simultaneously reducing NAD⁺ to NADH. The mechanism is reversible with a pH of less than 10. The NADH is then utilized by the diaphorase to convert the tetrazolium salt to its corresponding formazan, which can be seen with the eye.

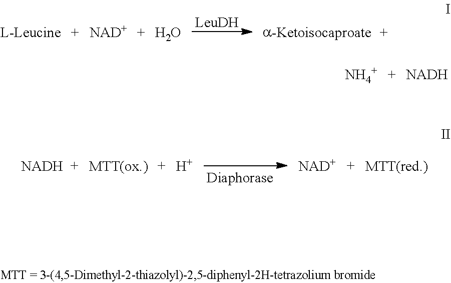

In one non-limiting embodiment, Membrane-3 also contains a buffer and stabilizing agents.

Membrane-3 is the color generating reagent layer of the test strip. The cell-separated biological solution containing BCAAs migrates to Membrane-3. Membrane-3 comprises surfactant(s), buffer, electron mediator(s), and an indicator such as, but not limited to, a tetrazolium salt. Membrane-3 may further be comprised of bio-active components generating signal, via color. In the presence of an electron mediator and indicator such as a tetrazolium salt, the end color intensity or rate of color development is proportional to the BCAA concentration in the biological sample.

Suitable electron mediators include diaphorase, which can be used in the reduction of tetrazolium salts. In addition, non-enzymatic electron transfer agents such as phenazine methosulfate (PMS), phenazine ethosulfate (PES), 1-Methoxy-5-methylphenazinium methylsulfate (1-methoxy PMS), or 1-Methoxy-5-ethylphenazinium ethylsulfate (1-methoxy PES) can be used. In one non-limiting embodiment, diaphorase is used in tandem with l-Methoxy-5-ethylphenazinium ethylsulfate (1-methoxy PES). Reaction kinetics and stability are the primary factors for selecting an electron mediator. PMS is, however, more sensitive to light than enzyme-based mediators. Diaphorase is more stable and, for that reason, is preferred when the cofactor is NAD⁺ or NADP⁺.

In one non-limiting embodiment, Membrane-3 is positioned over a light emitting diode with a wavelength for the detection of BCAAs between 400 and 700 nm. The kinetic or end-color intensity of the BCAAs can be measured by reflectance determination via a handheld meter. In another non-limiting embodiment, the Membrane-3 is positioned under a camera to image the end color development using RBG values, HVA values, or pixel count via a photometric image. The meter (or reader) contains software, which quantifies BCAAs in the range of 0 to 3.0 millimolar concentrations in biological fluid. Using a temperature correction algorithm, the meter can compensate for rate changes due to changes in testing temperatures.

These test strips can be incorporated into kits and assays for the quantitative determination of BCAAs in a biological fluid. In one non-limiting embodiment, a plurality of test strips is provided in a vial or in a kit or assay of the present invention further comprising alcohol wipes and/or a plurality of lancets and/or controls comprised, for example, of a polymeric solution spiked with BCAAs.

The test strips can also be used in methods for identifying and monitoring individuals with disorders relating to deficiency or excess in BCAAs and monitoring BCAA levels in such individuals.

The test strips, assays, kits, and methods of the present invention provide an analytical range of branched-chain amino acids from 0 to 3.0 millimolar concentrations.

In practice, the test strips, kits, assays, and methods of the present invention provide a point-of-care test useful by both clinicians and patients for diagnosis and monitoring of metabolic disorders associated with the deficiency or accumulation of BCAAs. BCAA assessment is a critical parameter for pre- and post-assessment in dietary restrictions and BCAA supplementation as well as upon the administration of certain medications. The test strips, kits, assays, and methods of this invention can be used for both initial diagnostic determination of the genetic disorder of MSUD and/or for monitoring purposes for patients on restricted diets, those receiving medication, as well as individuals testing before, during, and after athletic training. The assay can be performed with a volume of blood from fingerstick, which is less than 20 μL. This allows ease of use for the patient as well as the utilization of the test strips, kits, and assays in more rural or home settings.

Calibration of whole blood, urine, saliva samples can be obtained by spiked and/or modified whole blood reference samples. All samples are sent out to or received from reference laboratories to obtain BCAA millimolar concentrations via IEC or MS/MS. Samples of known reference values are assayed via the test strips and the percent reflectance/RBG/Pixel Count/HVA for each sample is recorded. These samples encompass the analytical range of BCAA in plasma, urine, or saliva.

For the millimolar BCAA concentrations obtained, a curve set is programmed into calibration software where Percent Reflectance/RGB Values/Pixel Count/HVA obtained by the meter (reader) software equals millimolar or mg/dL BCAA values from the laboratory reference. These values can be corrected for temperature as follows: (units BCAA)×TFC where TFC=Temperature Correction Factor.

The following non-limiting examples are provided to further illustrate the present invention.

### EXAMPLES

Two significant considerations in the present invention include: (1) the lowest concentration level at which the BCAA can be detected with high degree of reliability; (sensitivity); and (2) the range between the lowest analytical limit and the upper analytical limit (dynamic range).

In order to evaluate the BCAA assay of the present invention, titrations of spiked BCAA are prepared and evaluated by the said method.

### Example 1: Determination of BCAA in Charcoal Filtered Plasma (Delipidized Plasma)

An experiment was performed to investigate whether the invention described above is linear through the proposed analytical range 0 to 3.0 millimolar BCAAs in plasma.

For this experiment, the test strip contained: Layer-1: A spreading layer provided by Saati Tech, Hyphil 105/52 and three membranes additional to the spreading layer.

Membrane-1 is a blood separation membrane, CytoSep 1660 (Ahlstrom). This first membrane is impregnated with working solution-1 containing 0.1% Chitosan (Polysciences, Inc.), 0.2% Bovine Serum Albumin (Alfa Aesar), 20 mM HEPES buffer (TCI America) adjusted to pH 6.8, and 50 mM Sodium Chloride (Santa Cruz Biotechnology). This membrane is immersed in the working solution-1 and dried at 50° C. for 30 minutes.

Membrane-2 was comprised of a 1.2 μm Polyethersulfone membrane (GVS). Membrane-2 was impregnated with working solution-2 comprising 50 mM 2-Amino-2-methyl-1-propanol hydrochloride, pH 10.4 (Carbosynth), 0.2% Triton X-100 (Santa Cruz Biotech) and 0.2% polyvinyl alcohol (TCI America). This membrane was immersed in working solution-2 and dried at 50° C. for 20 minutes.

For Membrane-3, the reagent is Polyethersulfone 0.2 μm (GVS). Membrane-3 was impregnated with working solution-3 comprising 7 mg/g of beta-Nicotinamide adenine dinucleotide (Alfa Aesar), 5 mg/g of Thiazolyl blue tetrazolium bromide (Carbosynth), Diaphorase 150 Units/gram (Toyobo), and 300 units/gram Leucine dehydrogenase (Toyobo). The mordant used to immobilize the bioactive components was Plasdone™ K-29/32 (Ashland).

Delipidized plasma was purchased for spiking (Biocell). Deplididized plasma removes all endogenous analytes in plasma and can be used for spiking.

Stock solution-1 was delipidized plasma containing no branched-chain amino acids. Stock solution-2 was delipidized plasma containing 268 mg/dL of L-Leucine.

Working solutions: BCAA-free delipidized plasma was combined serially with the spiked delipidized plasma. Concentrations of 0, 5, 10, 20, 30, and 40 mg/dL L-Leucine were created and sent for mass spec analysis.

Twenty microliters of each sample was dispensed onto each test strip and the percent reflectance was recorded using a commercially available portable spectrophotometer (Konica Minolta CM-2600d). Each sample was assayed in quadruplicate.

Values obtained from the reference mass spec method were plotted against the percent reflectance recorded from the test strip.

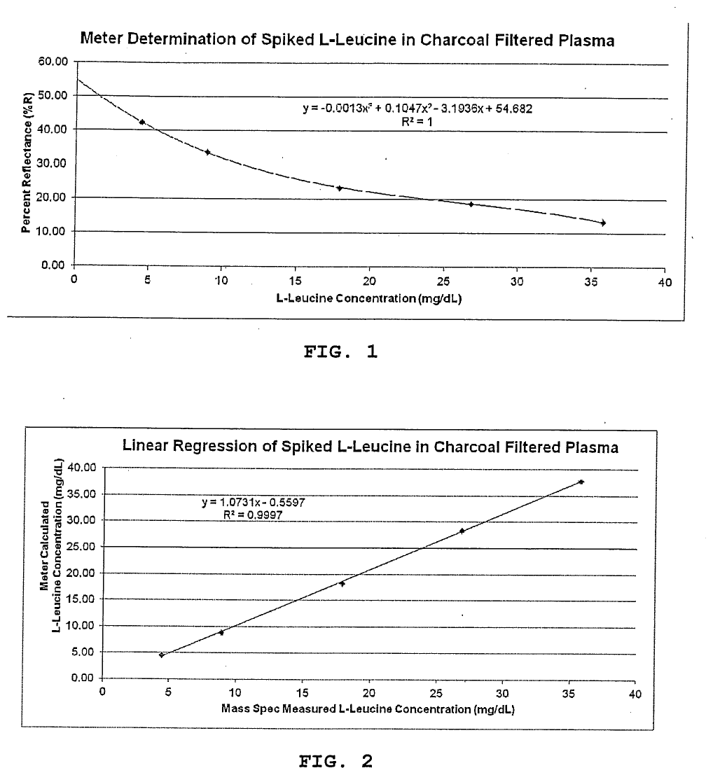

A 3^{rd} order polynomial of MS/MS concentration (mg/dL L-Leucine) versus the Percent Reflectance obtained is depicted in FIG. 1.

A first order linear regression of MS/MS concentration (mg/dL L-Leucine) versus interpolated mg/dL L-Leucine from the measured Percent Reflectance is depicted in FIG. 2.

Table 1 lists the precision of four replicate values and bias against the Mass Spec values.

TABLE 1
Percent Reflectance at Lambda Max

| L-Leucine | Rep | Rep | Rep | Rep | | Std. | % |
|---|---|---|---|---|---|---|---|
| (mg/dL) | 1 | 2 | 3 | 4 | Average: | Dev: | CV: |
| 0 | 77 | 73 | 75 | 75 | 75.00 | 2.00 | 2.67 |
| 4.47 | 42 | 42 | 43 | 43 | 42.33 | 0.58 | 1.36 |
| 8.93 | 33 | 33 | 35 | 33 | 33.67 | 1.15 | 3.43 |
| 17.87 | 23 | 23 | 24 | 24 | 23.33 | 0.58 | 2.47 |
| 26.8 | 19 | 18 | 19 | 19 | 18.67 | 0.58 | 3.09 |
| 35.73 | 13 | 13 | 14 | 15 | 13.33 | 0.58 | 4.33 |

| Meter Percent | Mass Spec L-Leucine | Meter L-Leucine | |
|---|---|---|---|
| Reflectance: | (mg/dL): | (mg/dL): | Bias: |
| 42.33 | 4.47 | 4.49 | 0.02 |
| 33.67 | 8.93 | 8.88 | 0.05 |
| 23.33 | 17.87 | 18.31 | 0.44 |
| 18.67 | 26.8 | 28.36 | 1.56 |
| 13.33 | 35.73 | 37.82 | 2.09 |

| | |
|---|---|
| Total Bias: | 4.16 |

Bias can be eliminated through calibration of the meter.

1: A test strip for the quantitative determination of one or more branched-chain amino acids in a biological samples, said test strip comprising:
(a) a spreading layer capable of distributing or metering the biological sample evenly across the surface of a primary membrane;
(b) a primary membrane comprising a blood separation membrane or preconditioning membrane containing hemagglutinating agents, buffer, and/or surfactants, said primary membrane being in direct contact with the spreading layer so that a biological sample applied to the test strip flows transversely across the primary membrane;
(c) a secondary membrane comprising a surfactant, a preconditioning buffer, mordant, and bioactive components for the quantitative determination of one or more branched-chain amino acids in biological fluids, said secondary membrane being in contact with the primary membrane so that the biological fluid migrates vertically or horizontally from the primary membrane into the secondary membrane; and
(d) a tertiary membrane or reagent membrane comprising a surfactant, an electron mediator, nicotinamide adenine dinucleotide, an indicator, and a bio-active enzyme capable of generating a signal which is proportional to the branched-chain amino acid concentration in the biological sample, said tertiary membrane being in direct contact with the secondary membrane.

2: The test strip of claim 1 wherein the biological sample is whole blood, urine, or saliva.

3: The test strip of claim 1 wherein the biological sample is whole blood and the primary membrane comprises one or more hemagglutinating agents.

4: The test strip of claim 1 wherein the branched chain amino acid is L-Valine, L-Leucine, L-Isoleucine and/or L-Alloisoleucine

5: A kit for the quantification of branched-chain amino acids in biological fluids, said kit comprising the test strip of claim 1.

6: A method for quantifying branched-chain amino acids in a biological sample, said method comprising applying a biological sample to the spreading layer of the test strip of claim 1 and reading the results of the test strip which quantify branched-chain amino acids in the biological sample.

7: The method of claim 6 further comprises obtaining the biological sample from the patient.

8: The method of claim 6 wherein the biological sample is whole blood, saliva, or urine.
